(19) United States
(12) Patent Application Publication
(10) Pub. No.: US 2006/0259035 A1
(43) Pub. Date: Nov. 16, 2006

(54) Method and Apparatus for Performing a Surgical Procedure

(76) Inventors: Camran Nezhat, Woodside, CA (US); Roger A. Stern, Cupertino, CA (US); Joseph Eder, Los Altos, CA (US); Peter Seth Edelstein, Menlo Park, CA (US)

Correspondence Address: GLENN PATENT GROUP, 3475 EDISON WAY, SUITE L, MENLO PARK, CA, 94025, US

(21) Appl. No.: 11/382,680

(22) Filed: May 10, 2006

Related U.S. Application Data
(60) Provisional application No. 60/680,937, filed on May 12, 2005.
(60) Provisional application No. 60/725,720, filed on Oct. 11, 2005.

(51) Int. Cl. A61B 18/14

(52) U.S. Cl. 606/50

(57) ABSTRACT

One method for performing procedures, such as vaginal hysterectomies, comprises engaging first and second energy transmitting elements against a lateral side of a uterus. The first and second energy transmitting elements are positioned against opposed surfaces of a tissue mass extending from and including a fallopian tube or round ligament to a tip of a cervix. Third and fourth energy transmitting elements are positioned against another lateral side of the uterus and against opposed surfaces of another tissue mass extending from and including another fallopian tube or round ligament to the tip of the cervix. Radio frequency or other high energy power is applied through the energy transmitting elements to the tissue masses. The power is applied for a time and in an amount sufficient to coagulate and seal the tissue masses within the energy transmitting elements. The coagulated tissue masses are then resected and the entire uterus removed.

### CROSS REFERENCE TO RELATED APPLICATIONS

This application derives its priority from U.S. Provisional Patent Application Ser. Nos. 60/680,937, filed May 12, 2005 and 60/725,720, filed Oct. 11, 2005, each of which is incorporated herein in its entirely by this reference thereto.

### BACKGROUND OF THE INVENTION

1. Field of the Invention

The invention relates generally to organ resection, and more particularly to methods and devices, for example, for surgical removal of the female uterus or hysterectomy.

2. Description of the Background Art

Hysterectomy may involve total or partial removal of the body and cervix of the uterus. Hysterectomy next to the caesarian section procedure is the most common surgical procedure performed in the United States. By the age of sixty, nearly one in three American women will have undergone hysterectomy. It is estimated that over a half million women undergo hysterectomy each year in the United States alone. The costs related to performing hysterectomies has burdened the United States healthcare system on the order of billions of dollars annually.

A majority of hysterectomies are performed by an open abdominal surgical procedure as surgeons have the most experience with this approach. An open abdominal surgical route allows the surgeon to easily view the pelvic organs in a larger operating space and also allows for removal of a large sized uterus or other diseased organs or tissue, such as the ovaries fallopian tubes, endometriosis, adenomyosis, and the like. However, open abdominal hysterectomy also suffers from several drawbacks. For example, the surgical procedure is often lengthy and complicated, requiring longer anesthesia periods and the increased risk of postoperative complications. Patients also suffer from prolonged recovery periods, pain and discomfort, and large visible scarring on the abdomen. Further, increased costs are associated with an open abdominal approach, such as prolonged hospital stays.

Two other common surgical approaches to performing hysterectomies which are less invasive are vaginal and laparoscopically assisted vaginal hysterectomy. A vaginal hysterectomy, which is of particular interest to the present invention, involves a surgical approach through the vaginal tubular tract to gain access directly to the uterus. Hysterectomies may also be performed with a range of laparoscopic assistance. For example, this may include the usage of a laparoscopic viewing port in a hysterectomy where all other steps are completed vaginally. In another example, the hysterectomy may be completely performed laparoscopically including removal of the uterus through a laparoscopic port.

Vaginal hysterectomies are more advantageous than open abdominal hysterectomy procedures for a variety of reasons, including fewer intraoperative and postoperative complications, shorter hospitalizations, and potentially reduced healthcare costs. Earlier resumption of regular activity, lower incidences of fever, ileus, and urinary tract infections, and little to no visible external scarring to the patient are additional benefits afforded by vaginal hysterectomy. Unfortunately, less than a third of all hysterectomies are performed vaginally due to a lack of surgeon training, limited access of the uterus and surrounding tissue, and unsuitability of a patient's anatomy, for example a large uterus size, limited vaginal access, severe endometriosis, pelvic adhesions, and the like.

For these reasons, it would be desirable to provide improved methods and devices for performing such procedures as a hysterectomy. In particular, it would be desirable to provide improved methods and devices for performing surgical procedures that reduce procedure time and complexity, resulting in improved patient outcomes and overall cost savings to the healthcare system.

### BRIEF SUMMARY OF THE INVENTION

The invention provides, inter alia, improved methods and devices for performing such procedures as vaginal hysterectomies, and that reduce procedure time and complexity, resulting in improved patient outcomes and potentially increased cost savings to the healthcare system. In one embodiment, the invention offers most advantages when performing a procedure, such as a hysterectomy, through a vaginal approach as described herein, yet is easier for the average surgeon to perform. It will be appreciated, however, that the presently disclosed devices may be modified to allow, for example, the removal of the uterus via open abdominal hysterectomy, which is also within the scope of the invention. Additionally, laparoscopic visualization may be used to guide the procedures of the invention. Those skilled in the art will appreciate that, while the invention is discussed in detail in connection with procedures performed on the uterus, i.e. a hysterectomy, other procedures are equally suited for application of the invention thereto. Accordingly, the invention applies equally to such other procedures and is not limited to the examples provided herein.

In one aspect of the invention, a method for performing a procedure, such as a hysterectomy, in a patient comprises engaging first and second energy transmitting forceps jaws against each of the two lateral sides of an organ or tissue, e.g. a uterus. In one embodiment, first and second energy transmitting elements are positioned against opposed surfaces of a tissue mass between a fallopian (uterine) tube and/or round ligament of the uterus and the cervix. Energy is applied through the energy dispersing elements to the tissue mass for a time and in an amount sufficient to coagulate and seal the tissue mass between the energy transmitting elements. Tissue along a plane within the coagulated tissue mass is then resected and the uterus removed. Removal of the fallopian tube(s) and/or ovary(ies) is an optional variation of the methods of the invention and may be determined by a distal most location of the energy transmitting elements. For example, if the fallopian tube(s) are not resected in the event that the fallopian tube(s) and potentially the ovary(ies) are to be removed along with the uterus, the distal most positioning of the energy transmitting elements extend from and include a suspensory ligament of the ovary(ies) and/or round ligament(s) below the fallopian tube(s). Still further, the fallopian tube(s) and potentially the ovary(ies) may be removed in a separate procedure using conventional vaginal or laparoscopic techniques.

In this embodiment, the invention avoids heating or ablation of the entire uterus. Instead, the invention focuses on surgically dividing, ligating, and severing the blood vessels, associated ligaments that support the uterus, and optionally the fallopian tube(s) and ovary(ies). This coagulates and seals off the entire blood supply to the uterus to effectively achieve hemostasis, i.e. cessation of bleeding, which is of major concern in removal of an organ or tissue, such as the uterus. This frees up the uterus for subsequent removal through the vaginal opening, as described in more detail below.

The first and second energy transmitting elements of a first jaw are preferably introduced through at least one small vaginal incision, possibly two small vaginal incisions, prior to engaging the energy transmitting elements against opposed tissue surfaces. Engaging generally comprises advancing the first and second energy transmitting elements up to or past the round ligament or fallopian tube. The first and second energy transmitting elements are then laterally pulled inward towards the uterus. The tissue mass therebetween is then compressed by clamping down on the first and second energy transmitting elements. In one embodiment, the first energy transmitting element spans a surface area of about 5 cm² to 10 cm², against a first tissue surface and the second energy transmitting element spans an area of 5 to 10 cm², against a second tissue surface. Typically, electrodes may each span a surface area between ½-10 cm², although in some embodiments, each electrode may comprise two or more elements, in which case each element may be less than 1 cm². For example, an electrode may be bifurcated longitudinally to define a channel therebetween along which a blade may pass, as discussed in greater detail below.

The introduction and engagement of the first and second energy transmitting elements may be viewed and guided with a laparoscope.

Third and fourth energy transmitting elements of a second jaw may either be introduced simultaneously with the first jaw as components of an integrated assembly, or sequentially through one or possibly two other small incisions in the vaginal wall, and advanced up to or past another round ligament or fallopian tube. The third and fourth energy transmitting elements are then laterally pulled inward against another lateral side of the uterus. The third and fourth energy transmitting elements are then clamped against opposed surfaces of another tissue mass extending between another fallopian tube or round ligament and the cervix so as to compress the another tissue mass therebetween. The third energy transmitting element spans a surface area of 5 cm² to 10 cm², against a third tissue surface and the fourth energy transmitting element spans an area of 5 to 10 cm², against a fourth tissue surface. Typically, electrodes may each span a surface area between ½-10 cm². Alternatively, electrodes comprised of multiple elements may have a surface area per element of less than 1 cm².

Again, the introduction and engagement of the third and fourth energy transmitting elements may be viewed and guided with a laparoscope. Additionally, a centering post may be inserted into the uterus and located parallel to and between the first and second jaws to allow the surgeon to maneuver the uterus externally. This, in turn, ensures proper viewing and positioning of the first and second jaws along lateral sides of the uterus, wherein all connective tissues and blood vessels are entrapped.

Once properly positioned, the first and second energy transmitting elements of the first jaw may be connected to the third and fourth energy transmitting element of the second jaw so as to form a single forceps unit if not previously introduced as an integrated assembly. Thereafter, energy may be delivered through the first and second energy transmitting elements of the first jaw to the tissue mass on the lateral side of the uterus and through the third and fourth energy transmitting elements of the second jaw to another tissue mass on another lateral side of the uterus. Optionally, the first and second jaw assemblies may be engaged and/or energized independently. Power is applied for a time and in an amount sufficient to coagulate the tissue within the first and second jaws to seal off the vessels supplying blood to the uterus and to prevent bleeding and free up the uterus for removal. Circuitry within the power supply may be used to detect appropriate and safe energy levels required to complete vessel sealing, discontinue energy delivery, and enable severing of the tissue. This procedure may be performed on both of the two lateral sides of the uterus simultaneously or in succession. The tissue masses engaged by the first and second forceps jaws comprise at least one of a broad ligament, facial plane, cardinal ligament, fallopian tube, round ligament, ovarian ligament, uterine artery, and any other connecting tissue and blood vessels. Sealing of the tissue masses by high energy and pressure from compression of the first and second forceps jaws results in elimination of the blood supply to the uterus to achieve hemostasis. Resecting comprises cutting coagulated tissue along a lateral plane on each side of the uterus. The uterus may then removed vaginally from the patient with the first and second forceps jaws or by other means, such as tensile extraction of the uterus with forceps or using a loop of suture that is applied through a portion of the cervix.

A variety of energy modalities may be delivered to the energy transmitting elements. Preferably, radio frequency power is delivered to electrode energy transmitting elements. For example, a conventional or custom radio frequency electrosurgical generator may be provided for delivering radio frequency power to the electrode elements. Treatments according the invention are usually effected by delivering radio frequency energy through the tissue masses in a bipolar manner where paired treatment electrodes, e.g., first and second electrode elements or third and fourth electrode elements, are employed to both form a complete circuit and to heat tissue therebetween uniformly and thoroughly. The paired electrode elements use similar or identical surface areas in contact with tissue and geometries so that current flux is not concentrated preferentially at either electrode relative to the other electrode. Such bipolar current delivery is to be contrasted with monopolar delivery where one electrode has a much smaller surface area and one or more counter or dispersive electrodes are placed on the patient's back or thighs to provide the necessary current return path. In the latter case, the smaller or active electrode is the only one to effect tissue as a result of the current flux which is concentrated thereabout. It will be appreciated, however, that other energy forms, such as thermal energy, laser energy, ultrasound energy, microwave energy, electrical resistance heating, and the like may be delivered to the energy transmitting elements for a time and in an amount sufficient to seal the vessels in the region. It will further be appreciated that depending upon the energy source, the second energy transmitting element may be an inactive or a return electrode, as opposed to being an active element.

In another aspect of the invention, electrocautery surgical tools for performing a procedure, such as a hysterectomy are provided. One tool comprises a first jaw having first and second jaw elements. A first energy transmitting element is disposed on the first jaw element and a second energy transmitting element is disposed on the second jaw element. The first and second energy transmitting elements are positionable against a lateral side of a uterus and against opposed surfaces of a tissue mass extending between, and including, a fallopian tube or round ligament and the cervix of the uterus. As described above, distal placement of the energy transmitting elements may be varied to also allow for removal of the fallopian tube(s) and/or ovary(ies). A handle is coupled to a proximal end of the first jaw. An electrical connector, or electrical cable and connector, is coupled to a proximal end of the handle for electrical connection to a radio frequency or other high energy electrosurgical generator, as described above.

The tool may also comprise a second jaw having third and fourth jaw elements. A third energy transmitting element is disposed on the third jaw element and a fourth energy transmitting element is disposed on the fourth jaw element. The third and fourth energy transmitting elements are positionable against another lateral side of the uterus and against opposed surfaces of another tissue mass extending between another fallopian tube or round ligament and cervix. The first and second jaws may also connect to one another via a joint mechanism to form a single forceps unit. Preferably, the gynecological tools, or portions thereof, of the invention are single use sterile, disposable surgical forceps.

The energy transmitting elements may take on a variety of forms, shapes, and sizes. The energy transmitting elements in this embodiment are preferably electrodes designed to fit the lateral sides of the uterus. Additionally, the jaw elements and/or electrodes may be curved along portions thereof to accommodate the anatomical shape of the uterus. Generally, the electrode elements may comprise flat, planar elongate surfaces. Typically, several square centimeters of opposed tissue surface area may be spanned, and the tissue mass therebetween coagulated and sealed with the gynecological devices of the invention.

The surgical tool may also comprise at least one cutting blade recessed within at least one jaw element to allow for tissue resection. The blade may movably traverse a longitudinal channel defined by pairs of electrode elements, as discussed above. The blade may comprise a variety of configurations, including a flexible blade, a cutting wheel, a v-shaped cutter, or a linkage blade, as will be described in more detail below. For safety purposes, a blade guide stop or blade interlock may be coupled to the blade so that the blade is not inadvertently released during the procedure, particularly prior to tissue desiccation. The surgical tool may also comprise at least one trigger mechanism coupled to the handle. For example, actuation of a first trigger clamps the first and second jaw elements together, which triggers the initiation of radio frequency power application. Actuation of a second trigger allows for tissue resection once complete tissue mass coagulation and sealing is verified. In such an embodiment, a change in impedance, current, or voltage is measured to verify that tissue mass coagulation and sealing is completed to prevent premature tissue resection. Further, an audible alarm may be sounded or a visual alarm displayed indicating complete tissue mass coagulation and sealing.

### BRIEF DESCRIPTION OF THE DRAWINGS

FIG. 1 illustrates a simplified frontal view of a uterus and its attaching structures;

FIG. 2 illustrates a partial simplified frontal view of a uterus with an electrocautery surgical tool constructed in accordance with the invention and positioned along a lateral side of the uterus according to the invention;

FIGS. 3A through 3F illustrate an exemplary method of the invention for performing a hysterectomy through a laparoscopically guided vaginal approach;

FIG. 4A illustrates a perspective view of a single jaw element having an electrode disposed thereon, while FIG. 4B illustrates compression of a tissue mass between two jaw elements;

FIGS. 5A and 5B illustrate tissue resection with a cutting blade after tissue desiccation;

FIGS. 6A though 6C illustrate another embodiment of the cutting blade that may be employed with the surgical tool of the invention;

FIGS. 7A through 7C illustrate still another embodiment of the cutting blade that may be employed with the surgical tool of the invention;

FIGS. 8A and 8B illustrate deployment of a device in accordance with the invention in connection with an abdominal incision;

FIG. 9 illustrates deployment of a device in accordance with the invention in connection with the division of a complex tissue sheet; and

FIG. 10 illustrates deployment of a device in accordance with the invention in connection with the division of an organ or tissue structure.

### DETAILED DESCRIPTION OF THE INVENTION

The invention provides methods and devices for performing such procedures as vaginal hysterectomies. It will be appreciated however that application of the invention is not limited to removal of the uterus, but may also be applied for ligation of nearby structures such as the ovaries (oophorectomy), ovaries and fallopian tubes (salpingo-oophorectomy), fallopian tubes, uterine artery, and the like. It will further be appreciated that the invention is not limited to a vaginal approach, but may also allow for removal of the uterus via open abdominal hysterectomy, which is also within the scope of the invention. Additionally, laparoscopic visualization may be used to guide the procedures of the invention. Finally, the invention is likewise applied to other parts of the body in connection with other surgical procedures.

FIG. 1 illustrates a simplified frontal view of a uterus 10 comprising a body 11 and a cervix 14. Attaching structures of the uterus 10 include fallopian (uterine) tubes 12, ovaries 13 and ligaments thereof 16, round ligaments 18 of the uterus, ureters 20, and uterosacral and cardinal ligaments 22 of the cervical neck 14. The broad ligament 24 of the uterus 10 is also shown.

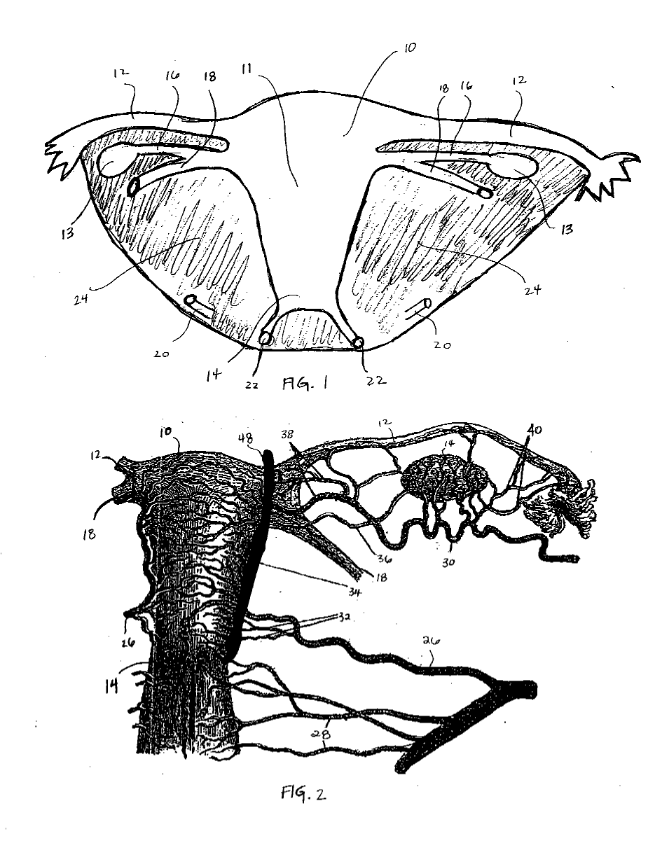

FIG. 2 shows the blood supply to the uterus 10, including the uterine artery 26, the vaginal arteries 28, and the ovarian artery 30, as well as branches to the cervix 32, body 34, round ligament 36, and fundus 38 of the uterus 10, and branches to the fallopian tube 40.

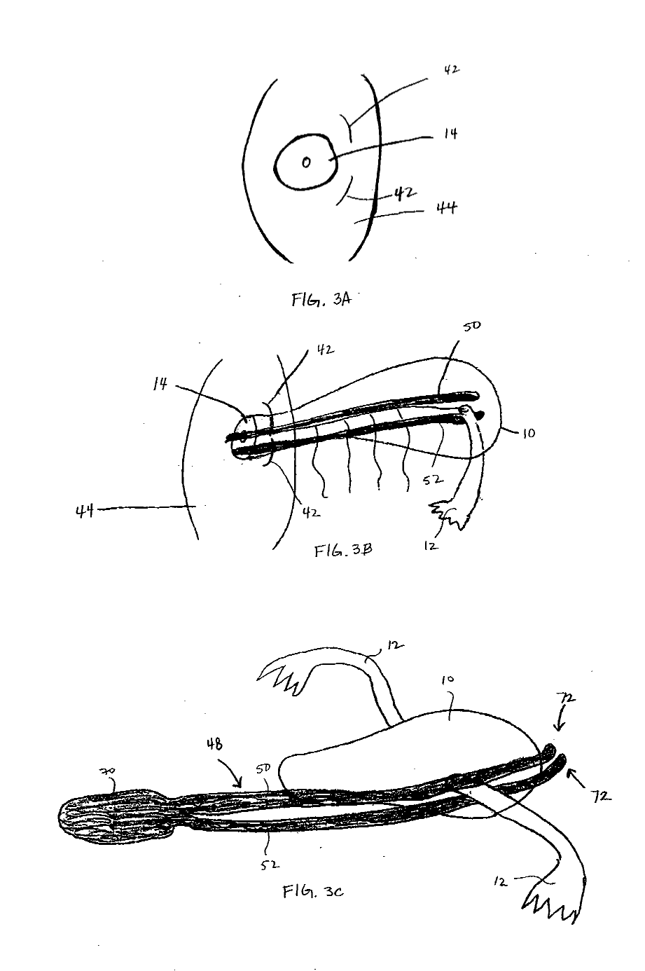

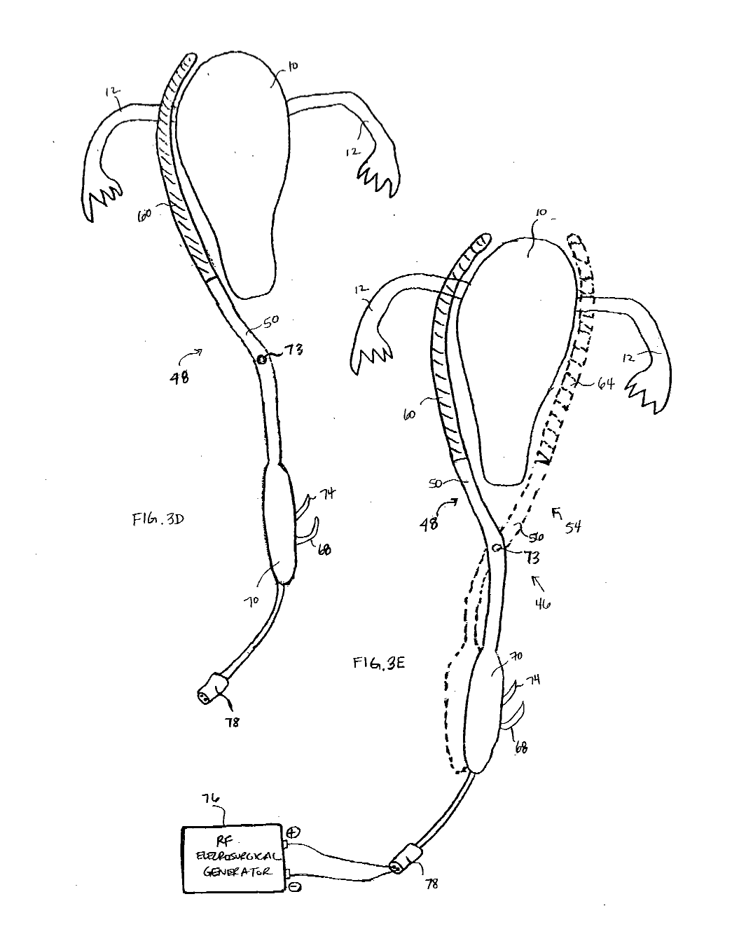

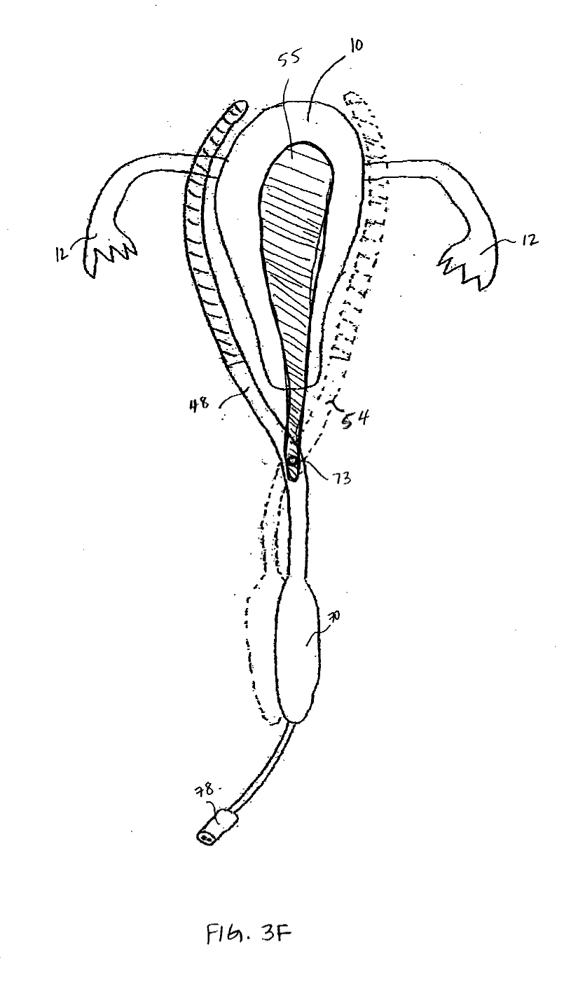

FIGS. 3A through 3E show, an exemplary method of the invention for performing a hysterectomy through a laparoscopically guided trans-vaginal approach. Initially, the patient is prepared per standard procedure as is known to those skilled in the art and a laparoscope inserted for visualization and guidance. FIG. 3A illustrates a view of the cervix 14 through the vaginal cavity 44 of the patient. One or two small incisions 42 are made through the vaginal wall 44 on the upper and lower sides of the cervix 14 to allow for introduction of the electrocautery surgical tool 46 of the invention into the pelvic cavity. It will be appreciated however that the procedures of the invention may be carried out via a single incision in the vaginal wall.

FIGS. 3B and 3E show, the electrocautery surgical forceps 46 of the invention which generally comprise a first jaw 48 having first and second jaw elements 50, 52 and a second jaw 54 having third and fourth jaw elements 56, 58. A first energy transmitting element 60 is disposed on the first jaw element 50 and a second energy transmitting element 62 is disposed on the second jaw element 52. Likewise, a third energy transmitting element 64 is disposed on the third jaw element 56 and a fourth energy transmitting element 66 is disposed on the fourth jaw element 58. The first and second jaws 48, 54 may be introduced either on a left hand side or right hand side of the patient at the same time or sequentially. As shown in FIG. 3B, the first jaw 48 is initially introduced in the right hand side of the cervix 14, wherein the first jaw element 50 is introduced through incision 42 in the vaginal wall and the second jaw element 52 is introduced through another incision 42 in the vaginal wall 44. These introductions may be performed simultaneously or sequentially.

The first and second jaw elements 50, 52 of the first jaw 48 are introduced and advanced possibly, but not necessarily, under laparoscopic visualization. The first jaw element 50 is above the broad ligament 24 and fascial plane while the second jaw element 52 is below the broad ligament 24 and fascial plane. If the fallopian tubes and ovaries are to be retained, the jaw elements 50, 52 are advanced until the first jaw 48 extends up to or past the round ligament 18 and the fallopian tube 12. The first and second jaw elements 50, 52 are then laterally moved inwards until they are against the body of the uterus 10 so as not to grasp the ureter 20 within the jaw elements 50, 52. At this point, the first and second energy transmitting elements 50, 52 are engaged against a lateral side of the uterus 10 and positioned against opposed surfaces of a tissue mass from the fallopian tube 12 to a portion of the cervix 14, as shown in FIG. 2. As described above, removal of the fallopian tube(s) 12 and/or ovary(ies) 13 is also within the scope of the methods of the invention. In such an embodiment where the fallopian tube 12 is not resected in the event that the fallopian tube 12 and, potentially, the ovary 13 are to be removed along with the uterus 10, the energy transmitting elements 50, 52 are positioned against opposed surfaces of a tissue mass extending from and including an ovarian ligament 16 and/or round ligament 18 below the fallopian tube 12 to a portion of the cervix 14.

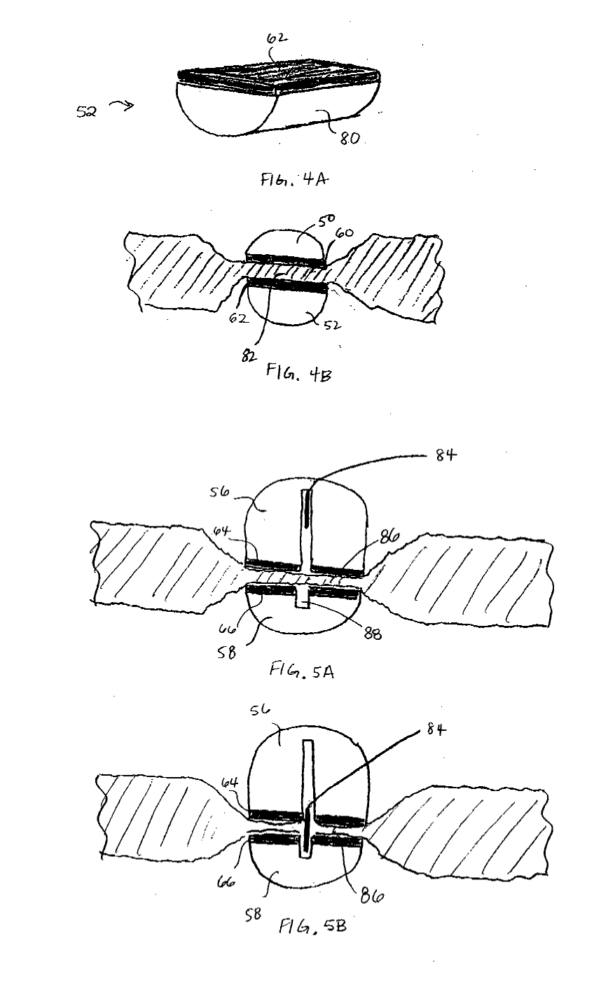

FIGS. 3C and 3D show, the entire tissue surface from the vaginal entrance adjacent to the cervix 14 all the way up to and past the round ligament 18 and optionally the fallopian tube 12, which is then grasped and compressed by clamping down on the first and second jaw elements 50, 52. This clamping motion of the jaw elements 50, 52 is depicted by arrows 72. A cross-sectional view of the tissue mass compressed between the first and second jaw elements 50, 52 is further illustrated in FIG. 4B. Typically, the first energy transmitting element 60 spans a surface area of 5 cm² to 10 cm², against a first tissue surface and the second energy transmitting element 62 spans an area of 5 to 10 cm², against a second tissue surface. More typically, the electrodes may each span a surface area between ½-10 cm², although in some embodiments, each electrode may comprise two or more elements, in which case each element may be less than 1 cm². For example, an electrode may be bifurcated longitudinally to define a channel therebetween along which a blade may pass, as discussed herein.

FIG. 3E shows third and fourth jaw elements 56, 58 of the second jaw 54 which may then be introduced in the left hand side of the cervix 14, wherein the third jaw element 56 is introduced through an incision in the vaginal wall and above the broad ligament 24 and the fourth jaw element 52 is introduced through another incision in the vaginal wall 44 and below the broad ligament 24. The third and fourth jaw elements 56, 58 are then advanced up to or past the left round ligament 18 and fallopian tube 12. The third and fourth jaw elements 56, 58 are then laterally pulled inward against the left lateral side of the uterus 10 so as not to grasp the ureter 20 within the jaw elements 56, 58. The third and fourth jaw elements 56, 58 are then clamped against opposed surfaces of another tissue mass extending from and including another fallopian tube 12 or round ligament 18 to a portion of the cervix 14 to compress the tissue mass therebetween. The third energy transmitting element 64 spans a surface area of 5 cm² to 10 cm², against a third tissue surface and the fourth energy transmitting element 66 spans an area of 5 to 10 cm², against a fourth tissue surface. Alternatively, electrodes comprised of multiple elements may have a surface area per element of less than 1 cm².

Again, the introduction and engagement of the third and fourth jaw elements 56, 58 may be viewed and guided with a laparoscope. Again, another option is to introduce jaws 48 and 54 simultaneously.

FIG. 3F shows, a centering post 55 which may be inserted into the uterus 10 and located parallel to and between the first and second jaws 48, 54 to allow the surgeon to maneuver the uterus externally in transverse or dorsal/ventral planes. This, in turn, ensures proper viewing and positioning of the first and second jaws 48, 54 along lateral sides of the uterus 10, wherein all connective tissues and blood vessels may be adequately entrapped. Once properly positioned, the central post 55 is locked into place with one or both sets of the electrocautery jaws 48, 54, for example via a joint mechanism 73. A cross sectional shape of the centering post 55 may comprise a tapered cylinder.

Referring back to FIG. 3E, all connecting tissues and blood vessels, including both right and left lateral sides of the cardinal ligament, broad ligament 24, uterine artery 26, and all the way up to the round ligament 18 and, optionally, the fallopian tubes 12 are grasped and compressed within the first and second jaws 48, 54. If not previously connected, once properly positioned, the first jaw 48 may be connected to the second jaw 54 via the joint mechanism 73 to form a single forceps unit 46 that may be easily manipulated by a surgeon. Thereafter, radio frequency power or other high energy modalities, as already described above, are delivered through the first and second energy transmitting elements 60, 62 of the first jaw 48 to the tissue mass on right lateral side of the uterus 10, and through the third and fourth energy transmitting elements 64, 66 of the second jaw 54 to another tissue mass on left lateral side of the uterus 10. Power is applied for a time and in an amount sufficient to coagulate the tissue within the first and second jaws 48, 54. Methods of the invention focus on surgically dividing and ligating the uterine arteries 26, round ligaments 18, and fallopian tubes 12. This coagulates and seals off the entire blood supply to the uterus 10 so as to achieve hemostasis effectively and free up the uterus 10 for subsequent removal through the vaginal cavity 44.

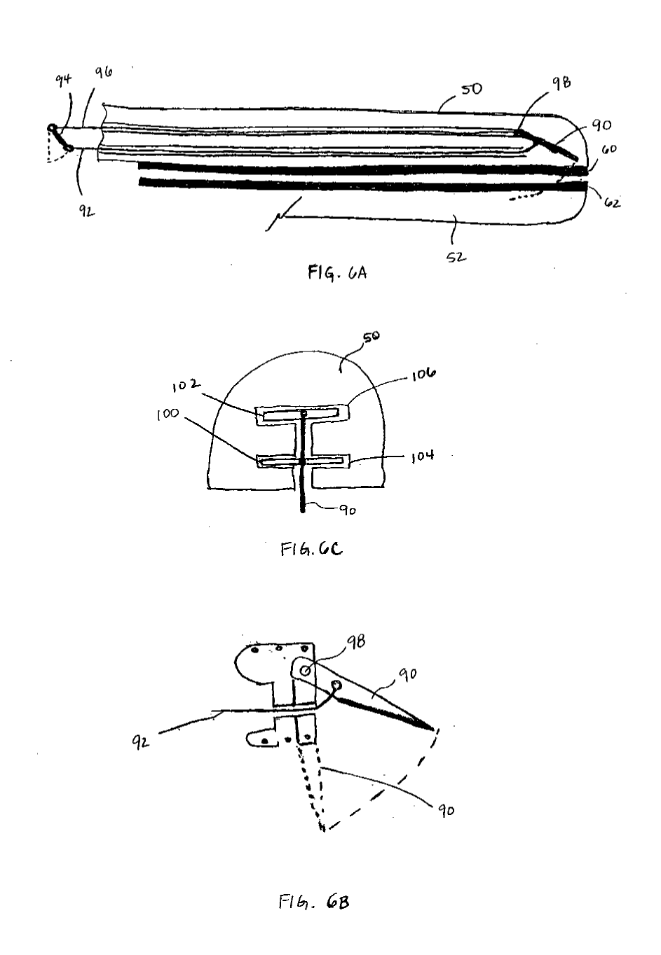

After sealing of the tissue mass by high energy and pressure from compression of the first and second forceps jaws 48, 54, the coagulated tissue may be cut along a lateral plane on each side of the uterus 10 by a variety of integrated cutting mechanisms, as described below with respect to FIGS. 5A though 7C. In lieu of secondary cutting mechanisms, the methods of the invention may alternatively comprise severing of the blood vessels and connective tissues of the uterus 10 by applying continuous or additional pressure to the first and second jaws 48, 54 post-electrocoagulation. For example, a secondary ridge-like device that does not penetrate and cut tissue prior to tissue cauterization may cut the more brittle cauterized tissue due to the additional compressive pressure exerted post-coagulation. Still further, resecting of the tissue may be carried out by increasing the energy density in the coagulated and sealed tissue mass by modifying energy transmission from a cautery mode to a cutting mode. In any embodiment, each half of the uterus 10 is freed from its surrounding attachments, including the fallopian tubes 12, round ligaments 18, uterine arteries 26, broad ligaments 24, cervical neck ligaments 22, and the like. The uterus 10 is then removed vaginally from the patient with the first and second forceps jaws 48, 54 or by other means of vaginal extraction. The laparoscope, if used, is then removed and the opening at the back of the vaginal cavity closed.

Such a vaginal hysterectomy results in numerous benefits. For example, procedure complexity is significantly reduced because the uterus is removed in one piece. Additionally, the time associated with such a procedure may be significantly shorter when compared to conventional hysterectomy procedures that require more than a hour of surgical time. This results in enhanced surgeon efficiency, improved patient outcomes, and overall cost savings to the healthcare system. Further, a surgeon with average skill may perform this procedure because laparoscopic visualization is used to guide the procedure.

A radio frequency electrosurgical generator 76 may be coupled to the forceps 46 via a multi-pin electrical connector 78 for delivering radio frequency power to electrode energy transmitting elements in a sufficient frequency range. Treatments according the invention are usually effected by delivering radio frequency energy through the tissue masses in a bipolar manner, where paired treatment electrodes are employed to both form a complete circuit and to heat tissue therebetween uniformly and thoroughly. For example, the first and third electrodes 60, 64 may be of one polarity (+) and the second and fourth electrodes 62, 66 may be of an opposite polarity (−) so that current flows between the first and second electrode pair 60, 62 and between the third and forth electrode pair 64, 66. The bipolar electrode elements heat the tissue masses to a sufficient temperature for a sufficient time period.

In some embodiments, a first trigger mechanism 68 may be coupled to a handle 70 of the forceps 46. Actuation of this first trigger mechanism 68 may clamp the jaw elements 50, 52, 56, 58 of the first and second jaws 48, 54 together and automatically trigger electrical circuitry that initiates the radio frequency power application though the energy transmitting elements 60, 62, 64, 66. This safety feature ensures that the tissue is properly positioned and engaged before it can be heated. Further, a change in impedance, voltage, or current draw (assuming constant voltage operation) may be measured by the circuitry/electronics of the power generator 76 to detect completion of the coagulation and sealing process. This feedback method confirms completion of coagulation before any tissue resection methods, as described above, can be undertaken. Actuation of a second trigger mechanism 74 coupled to the handle 70 or though increased pressure in the first trigger mechanism 68 may allow for tissue resection once complete tissue mass coagulation and sealing has been confirmed to prevent premature cutting. In such an embodiment, an audible alarm may be sounded or a visual alarm displayed, indicating complete tissue mass coagulation and sealing. The trigger system may be activated via solenoid activation of a pin which engages a linkage between the trigger and a cutting blade. A motor that advances the pin that engages the trigger can also be employed. Conversely, such solenoid or motor activation means advances a pin or linkage that removes a safety stop or brake that otherwise prevents the trigger mechanism from activating the cutting blade.

FIG. 4A illustrates a perspective view of the lower second jaw element 52 comprising the first energy transmitting element region 62 and an electrically insulating region 80 forming a support part of the jaw element 52. The coagulation zone of the compressed tissue mass 82, as illustrated in FIG. 4B, depends upon the geometry of the energy transmitting elements 60, 62. The energy transmitting elements preferably comprise electrodes that fit the lateral side of the uterus 10. Additionally, the jaw elements 50, 52, 56, 58 and/or electrodes 60, 62, 64, 66 may be curved along portions thereof to accommodate the anatomical shape of the uterus 10. Generally, the electrodes 60, 62, 64, 66 may comprise flat, planar elongate surfaces. Typically, several square centimeters of opposed tissue surface area may be spanned and the tissue mass therebetween coagulated and sealed with the gynecological devices of the invention.

FIGS. 5A and 5B illustrate tissue resection with a cutting blade 84 after tissue desiccation. FIG. 5A illustrates the third and fourth jaw elements 56, 58 of the second jaw 54, wherein the cutting blade 84 is recessed within the upper jaw element 56 in a retracted configuration. As shown in FIG. 5B, the cutting blade 84 is extended into a channel 88 of the lower jaw element 58 to allow for tissue resection once tissue desiccation 86 by the energy transmitting elements 64, 66 is completed. The cutting blade 84 in this embodiment comprises a flexible blade that is actuated by a pulling motion that moves it down and across the desiccated tissue 86 in a unidirectional saw-like motion along the entire length of the energy transmitting elements 64, 66. In one embodiment, the blade comprises a v-shaped cutter which defines a groove that captures the tissue as the blade is advanced longitudinally and that forces the captured tissue against a pair of cutting surfaces defined by the v-shaped cutter. In this embodiment, the energy transmitting elements are compound elements, divided by the recess for the cutting blade 84 in a first of the jaw elements 56 and by the channel 88 in a second of the jaw elements 58, respectively. In such embodiment, a total surface area of each compound energy transmitting element spans 5-10 cm², with each element of the compound element spanning a portion of the total surface area, e.g. 1.25-2.5 cm² or less.

The cutting blade 84 is guided by a number of diagonal slots (not shown) that are located at set intervals, e.g. several centimeters apart, along the length of the cutting blade 84. Pins placed in the slots that are fixed in the jaw element 56 serve as guides that limit the motion of the blade 84. As transverse motion is exerted on a proximal end of the blade 84, due to the diagonal slots, the blade 84 moves both backwards and down in single unidirectional sawing motion. The depth of blade exposure is in the range from about 1 mm to about 20 mm. Accordingly, the jaw elements 50, 52, 56, 58 should accommodate the blade depth.

FIGS. 6A through 6C illustrate a linkage blade 90 embodiment that may be employed with the surgical tool of the invention. FIG. 6A illustrates the first and second jaw elements 50, 52 of the first jaw 48, wherein the linkage blade 90 is recessed within the upper jaw element 50 in a retracted configuration. Pulling on a lower pull wire 92 brings the linkage 94 to a vertical position, as shown in broken line which, in turn, rotates the cutting blade 90 about an axle joint 98 to a vertical cutting position, as shown in broken line in FIG. 6B. Pulling on both the lower pull wire 92 and an upper pull wire 96 results in moving the lower and upper track sliders 100, 102 along the lower and upper pull wire tracks 104, 106 which, in turn, moves the cutting blade through the tissue that has been desiccated by the energy transmitting elements 60, 62, as shown in FIG. 6C.

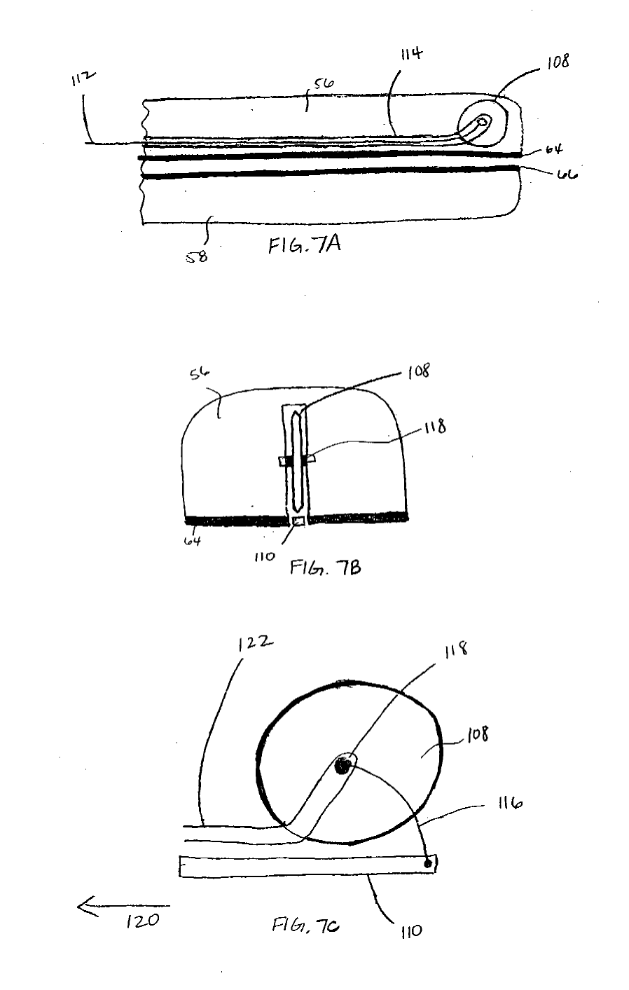

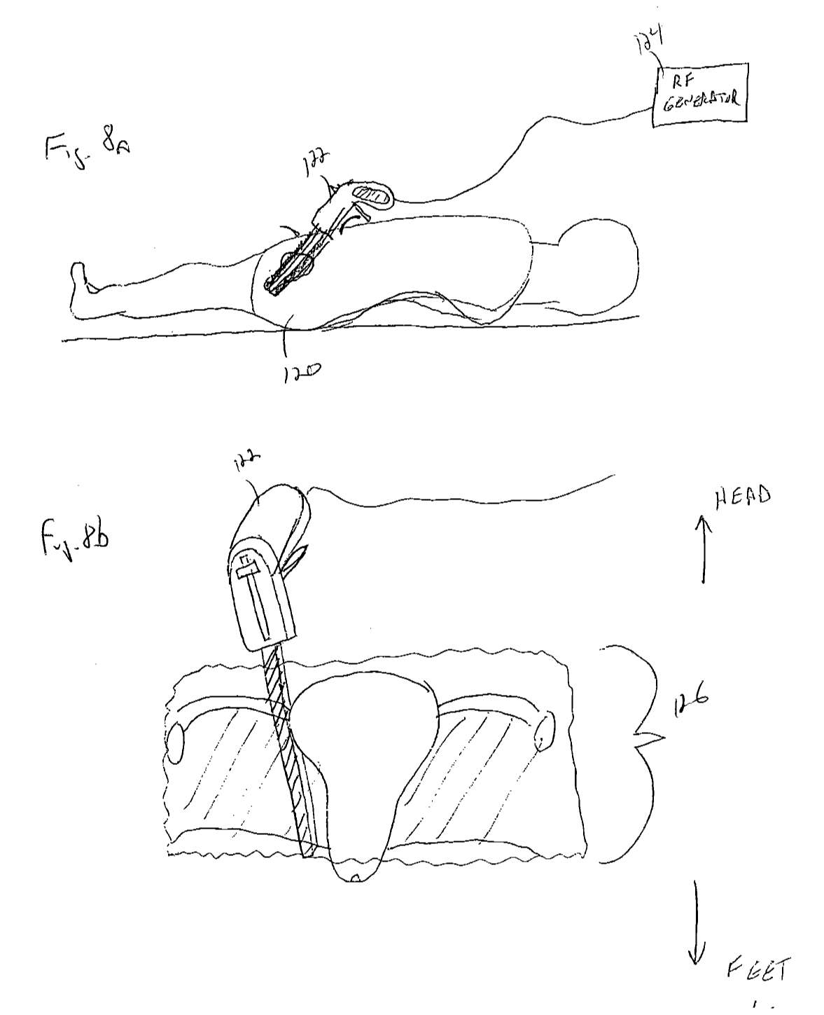

FIGS. 7A through 7C illustrate a cutting wheel 108 embodiment that may be employed with the surgical tool of the invention. FIG. 7A illustrates the third and fourth jaw elements 56, 58 of the second jaw 54, wherein the cutting wheel 108 is recessed within the upper jaw element 56 in a retracted configuration. In this embodiment, a pull wire 112 may roll the cutting wheel 108 down and across the desiccated tissue along channels 114 in the jaw elements 56, 58. As shown in FIG. 7B, a blade guide stop 110 may additionally be provided so that the cutting blade 108 is not inadvertently released during the hysterectomy, particularly prior to electrocautery completion. In such an embodiment, pulling back on the blade guide stop 110, as depicted by arrow 120, initially exposes the cutting wheel 108. A wire 116 attached to a distal end of the blade guide stop 110 and axle joint 118 of the cutting wheel 108 then pulls the cutting wheel 108 down and along the cutting wheel track 122.

It will be appreciated that the all the above depictions are for illustrative purposes only and do not necessarily reflect the actual shape, size, or dimensions of the forceps device 46.

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. For example, the methods and devices of the invention may be employed to remove the uterus via laparotomy, through an abdominal incision. Energy is applied until complete coagulation and vessel sealing is achieved. The coagulated tissue is then resected, freeing up the organ which may be removed through the abdominal incision.

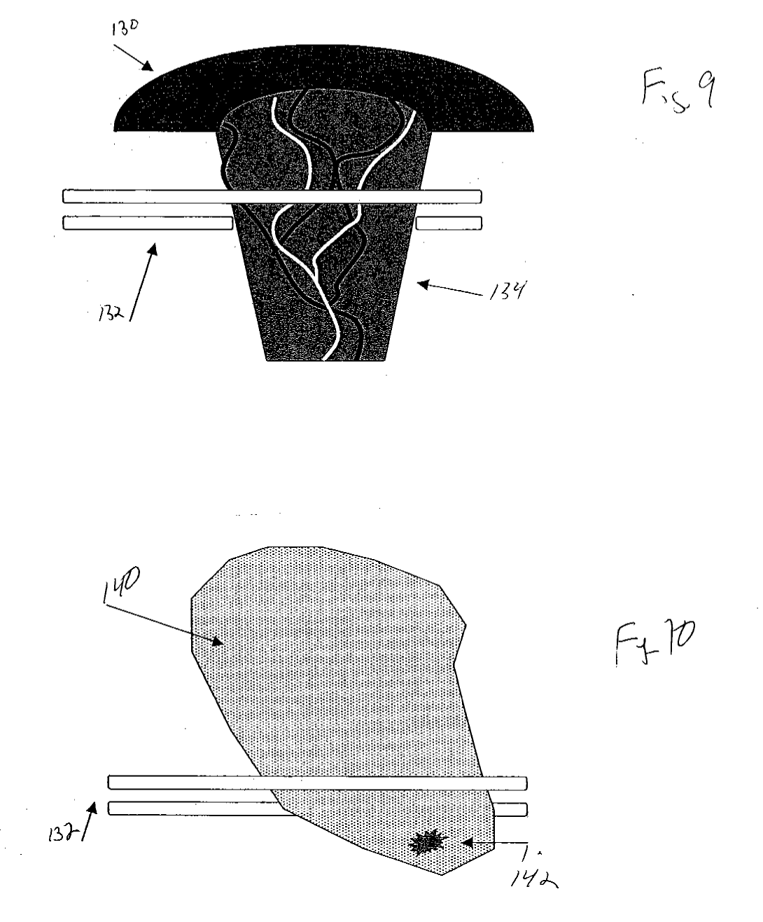

FIGS. 8A and 8B illustrate deployment of a device in accordance with the invention via an abdominal incision. Therefore, the above description should not be taken as limiting the scope of the invention, which is defined by the appended Claims.

FIG. 8A shows a side view of a deployment of a device 122 according to the invention for purposes of an abdominal incision into an individual 120. Also shown in FIG. 8A is the RF generator 124. FIG. 8B is a top view showing the deployment of the device 122 via an abdominal incision 126. Orientation of the individual's head and feet is indicated in FIG. 8B.

Resection of Complex Tissue Sheets

The following embodiment of the invention is based on the observation that numerous surgical procedures require division of long, complex sheets of tissue, composed of blood vessels, nerves, ligaments, fat, connective tissue, and additional critical structures. Routinely, these complex tissue sheets are divided via a long and repetitive process in which blood vessels and other critical structures, such as fallopian tubes, are first individually dissected free from surrounding tissues and subsequently individually divided and ligated. Next, the remaining connective tissue is divided, often in piece-meal fashion. As noted above, the entire process is time and labor-intensive. In addition, adjacent vital structures are repeatedly at risk for injury during the repeated dissection, division, and ligation procedures. Post-operatively, inflammation and necrosis within the suture-ligated tissues generate significant pain. The above-described inventive radio frequency energy (RF) power supply and platform of procedure-specific devices allows for the rapid, safe, and simple division of complex tissue sheets. The procedure-specific devices that may be provided with the invention share some of the features discussed above in connection with the preferred embodiment, including a handle and two blades, which can be opened to be placed across the tissue sheet in the manner analogous to scissors across paper, and enclosed, thereby capturing and containing a tissue sheet. The invention also comprises a long, narrow bi-polar electrode embedded into two blades, which cauterizes the contained tissue when RF is delivered from the power supply. The invention further may comprise either a mechanical scalpel or RF feature which allows for division of the cauterized tissue. Broadly, the invention comprising these elements cauterizes a complex tissue sheet and divides same in seconds, without the need for dissection or piece-meal division or ligation. The above embodiment concerning a hysterectomy is an example of this.

Further, with the invention, operative time and cost are reduced, and operative safety is improved because adjacent vital structures are only at risk for injury one time, during visualized placement of the device, and post-operative pain is reduced due to the absence of significant tissue inflammation and necroses when RF is used to divide tissue, as is supported in the medical literature.

The resection of all or part of an organ, such as the spleen, or tissue structure, such as a muscle, frequently involves a division of associated complex tissue sheets, including all vascular structures, lymphatics, nervous system tissue, connective tissue, adipose tissue, and the like. The complex tissue sheets associated with different organs are tissue structures in their composition. For example, the small bowel (duodenum, jejunum, and ileum) is supported by a complex tissue sheet, as is the small bowel mesentery, which includes arterioles and arteries, venules and veins, lymphatic vessels, and lymph nodes, microscopic nerve fibers, minimal adipose tissue, and avascular connective tissue. The omentum, on the other hand, contains a large volume of adipose tissue, a great number of emphatic vessels and lymph nodes, and numerous large arteries and veins. Thus, the power supply and device used to resect one organ or tissues structure, such as a small bowel, must differ from the power supply and device used resect a different organ or tissue structure, such as the omentum, in a number of characteristics including, but not limited to:
length of jaw;
shape of jaw;
clearance of jaw;
closure force jaw;
length of electrodes;
width of electrodes;
depth of recessing electrodes within one and both blades;
ergonomics of handle;
power supply voltage;
power supply delivered power;
tissue impedance threshold;
duration of RF delivery;
mechanical approach to tissue division; and
RF approach to tissue division.

In a variety of surgical procedures, procedure-specific surgical equipment as described above is used to divide complex tissue sheets. FIG. 9 is a diagram providing an example an ileal resection in which the complex tissue sheet is a small bowel mesentery. In FIG. 9, a representation is shown of the ileum and mesentery (with arteries, veins, lymphatics, connective, nervous, adipose tissue). The herein surgical device, in this embodiment comprising two blades, is placed across a complex tissue sheet (the mesentery). Such use of the herein described invention is application to resection of all or part of the following organs or tissue structures:
the esophagus;
the duodenum;
the jejunum;
the ileum;
the colon;
the rectum;
the stomach;
the spleen;
the kidney;
the omentum;
the pancreas;
the liver;
the lungs; and
muscular.

Resection of the Portion of an Organ and Tissue Structure

Different power supply and device characteristics are required in connection with the equipment used to divide different organs or tissue structures. For example, division of lung tissue must normally address hemostatic sealing of arterioles, venules, and capillaries, but must also abide closure of alveolar (microscopic air) sacs to limit or prevent post-resection air leak. However, the division of the pancreas must address cauterization of fatty glandular tissue and creation of the seal across the pancreatic duct. Thus, as with the approach to division of complex tissue sheets, the approach to division of organs and tissue structures also requires procedure-specific power supply and device features. Those skilled in the art will appreciate that the invention described above in connection with the performance of the hysterectomy is readily adapted for these procedures.

In a variety of surgical procedures, procedure-specific surgical equipment in accordance with the invention herein is used to divide the organs and tissues structures. FIG. 10 illustrates an example of a partial lung resection. In FIG. 10 a lung 140 shown having a pathological condition 142. The procedure is to divide a lung and remove the pathological section therefrom. To accomplish this, the herein disclosed surgical device, in this embodiment comprising two blades, is placed across the lung to effect organ division. Such use of the herein disclosed device is applicable to resection of part of the following organs with tissue structures:
the omentum;
the pancreas;
the liver;
the lung;
the muscular; and
skin and integument.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A method for performing a surgical procedure in a patient, the method comprising:
engaging first and second energy transmitting elements against a lateral side of an organ or tissue structure, wherein the first and second energy transmitting elements are positioned against opposed surfaces of a tissue mass;
applying energy through the energy transmitting elements to the tissue mass, wherein the energy is applied for a time and in an amount sufficient to coagulate and seal the tissue mass between the energy transmitting elements; and
resecting tissue along a plane within the coagulated tissue mass

2. The method of claim 1, wherein said surgical procedure comprises a hysterectomy; and where said organ comprises a uterus.

3. A method as in claim 2, wherein the first and second energy transmitting elements are positioned against opposed surfaces of a tissue mass between from and including a fallopian tube or round ligament and a cervix.

4. A method as in claim 2, wherein the first and second energy transmitting elements are positioned against opposed surfaces of a tissue mass extending from and including an ovarian ligament or round ligament to a tip of a cervix.

5. A method as in claim 2, wherein engaging comprises advancing the first and second energy transmitting elements up to or past a round ligament or fallopian tube, moving the first and second energy transmitting elements laterally inward towards the uterus, and compressing the tissue mass therebetween.

6. A method as in claim 1, wherein compressing the tissue mass comprises clamping the first and second energy transmitting elements together which triggers energy power application.

7. A method as in claim 1, wherein engaging comprises positioning the first energy transmitting element which spans a surface area of 1/2 -10 cm² against a first tissue surface and positioning the second energy transmitting element which spans an area of ½-10 cm² against a second tissue surface.

8. A method as in claim 1, wherein engaging comprises positioning the first energy transmitting element comprising at least two elements arranged to define a longitudinal gap therebetween and which spans a combined surface area of ½-10 cm² against a first tissue surface and positioning the second energy transmitting element comprising at least two elements arranged to define a longitudinal gap therebetween and which spans an area of ½-10 cm² against a second tissue surface.

9. A method as in claim 2, further comprising introducing the first and second energy transmitting elements of a first jaw through at least one vaginal incision prior to engaging the energy transmitting elements against opposed tissue surfaces.

10. A method as in claim 2, further comprising introducing the first and second energy transmitting elements of a first jaw through an abdominal incision prior to engaging the energy transmitting elements against opposed tissue surfaces.

11. A method as in claim 1, further comprising viewing and guiding the introduction and engagement of the first and second energy transmitting elements with a laparoscope.

12. A method as in claim 1, further comprising engaging third and fourth energy transmitting elements against another lateral side of said organ or tissue structure, wherein the third and fourth energy transmitting elements are positioned against opposed surfaces of another tissue mass.

13. A method as in claim 12, further comprising inserting a centering post into the uterus so as to allow for external maneuvering of the uterus.

14. A method as in claim 1, wherein the first and second energy transmitting elements of a first jaw are introduced simultaneously or sequentially with the third and fourth energy transmitting element of a second jaw.

15. A method as in claim 1, further comprising connecting the first and second energy transmitting elements of a first jaw to the third and fourth energy transmitting element of a second jaw so as to form a single unit.

16. A method as in claim 1, further comprising applying pressure to the first and second jaws so as to initiate energy delivery through the first and second energy transmitting elements of a first jaw to the tissue mass on the lateral side of said organ or tissue structure and through the third and fourth energy transmitting element of a second jaw to another tissue mass on another lateral side said organ or tissue structure of the uterus, wherein the energy is applied for a time and in an amount sufficient to coagulate and seal the tissue within the first and second jaws.

17. A method as in claim 1, further comprising measuring a change in impedance, voltage, power, energy, time, temperature or combination thereof, or current so as to verify complete tissue mass coagulation and sealing.

18. A method as in claim 1, further comprising sounding an audible alarm or displaying a visual alarm indicating complete tissue mass coagulation and sealing.

19. A method as in claim 1, further comprising triggering tissue resection once complete tissue mass coagulation and sealing has been verified.

20. A method as in claim 19, wherein resecting comprises cutting coagulated tissue along a lateral plane on each side of said organ or tissue structure.

21. A method as in claim 19, wherein resecting is carried out by applying continuous or additional pressure to the first and second jaws.

22. A method as in claim 19, wherein resecting is carried out by a cuffing blade.

23. A method as in claim 22, further comprising releasing a blade interlock prior to tissue resection.

24. A method as in claim 19, wherein in resecting is carried out by increasing the energy density in the coagulated and sealed tissue mass.

25. A method as in claim 1, further comprising removing at least a section of said organ or tissue structure from the patient with the first and second jaws.

26. A method as in claim 1, wherein sealing comprises closing a blood supply to said organ or tissue structure.

27. A method as in claim 2, wherein the tissue mass comprises at
least one of a broad ligament, facial plane, cardinal ligament, fallopian tube, round ligament, ovarian ligament, uterine artery, and vaginal tissue.

28. A method as in claim 1, wherein the energy comprises radio frequency energy, thermal energy, laser energy, ultrasound energy, microwave energy, or electrical resistance heating.

29. A method as in claim 28, wherein the second energy transmitting element comprises an inactive element or a return electrode.

30. A method as in claim 28, wherein the energy transmitting elements comprise electrodes and applying comprises delivering sufficient radio frequency power at a sufficient frequency.

31. A method as in claim 30, wherein radio frequency energy is delivered in a bipolar manner.

32. A surgical tool for performing a surgical procedure in a patient, the tool comprising:
a first jaw having first and second jaw elements, wherein a first energy transmitting element is disposed on the first jaw element and a second energy transmitting element is disposed on the second jaw element, the first and second energy transmitting elements being positionable against opposed surfaces of a tissue mass;
a handle coupled to a proximal end of the first jaw;
a connector coupled to a proximal end of the handle for electrical connection to an electrosurgical generator.

33. The tool of claim 32, wherein said surgical procedure comprises a hysterectomy.

34. A surgical tool as in claim 33, wherein the first and second energy transmitting elements are positionable against opposed surfaces of a tissue mass extending from and including a fallopian tube or round ligament to a tip of a cervix.

35. A surgical tool as in claim 33, wherein the first and second energy transmitting elements are positionable against opposed surfaces of a tissue mass extending from and including a round ligament or ovarian ligament to a tip of a cervix.

36. A surgical tool as in claim 32, further comprising a second jaw having third and fourth jaw elements, wherein a third energy transmitting element is disposed on the third jaw element and a fourth energy transmitting element is disposed on the fourth jaw element, the third and fourth energy transmitting elements positionable against another lateral side of said tissue mass.

37. A surgical tool as in claim 32, further comprising a centering post located parallel to and between the first and second jaws.

38. A surgical tool as in claim 32, wherein the energy transmitting elements comprise electrodes.

39. A surgical tool as in claim 38, wherein the electrodes are sized to fit the lateral side of the tissue mass.

40. A surgical tool as in claim 32, wherein the electrodes comprise elongate surfaces.

41. The surgical tool in claim 40, wherein the electrodes each comprise at least two elements arranged to define a longitudinal gap therebetween which defines a channel which a blade may longitudinally traverse.

42. A surgical tool as in claim 32, further comprising at least one blade recessed within at least one jaw element.

43. A surgical tool as in claim 4 2, wherein the blade comprises a flexible blade, a cutting wheel, a v-shaped blade, or a linkage blade.

44. A surgical tool as in claim 42, further comprising a blade guide stop coupled to the blade.

45. A surgical tool as in claim 32, further comprising at least one trigger mechanism coupled to the handle.

46. A surgical tool as in claim 32, wherein the connector provides electrical connection to a radio frequency electrosurgical generator.

47. A surgical tool as in claim 46, wherein the electrosurgical generator further comprises circuitry that detects a change in impedance, voltage, power, energy, time, temperature or combination thereof, or current so as to verify complete tissue mass coagulation and sealing.
